(19) United States
(12) Patent Application Publication
(10) Pub. No.: US 2024/0050672 A1
(43) Pub. Date: Feb. 15, 2024

(54) OXYGEN HOSE DISCONNECTION ALERT DEVICE

(71) Applicant: James Tyson Blackburn, Tulsa, OK (US)

(72) Inventor: James Tyson Blackburn, Tulsa, OK (US)

(21) Appl. No.: 18/234,244

(22) Filed: Aug. 15, 2023

Related U.S. Application Data
(60) Provisional application No. 63/398,162, filed on Aug. 15, 2022.

(51) Int. Cl. A61M 16/00; A61M 39/10

(52) U.S. Cl. CPC A61M 16/0051; A61M 16/0003; A61M 39/1055; A61M 2039/1005

(57) ABSTRACT

An oxygen hose disconnection alert device which connects to an oxygen supply line and notifies a patient or caretaker if the oxygen supply line becomes disconnected. The device may be used to couple two oxygen hoses together, to connect an oxygen hose to an oxygen supply source, or to connect an oxygen hose to an oxygen delivery device.

### CROSS REFERENCE

This application is based on and claims priority to U.S. Patent No. 63/398,162 filed Aug. 15, 2022.

### BACKGROUND OF THE INVENTION

### Field of the Invention

This invention relates generally to a device that alerts a user if an oxygen hose becomes disconnected from an oxygen source.

### Description of the Related Art

Oxygen hoses are commonly used to deliver supplemental oxygen to a patient or person in need of respiratory help. The lightweight tube is connected on one end to an oxygen supply, such as an oxygen tank or oxygen concentrator, and on the other end to an oxygen delivery device such as an oxygen mask or a nasal cannula. Oxygen flows from the oxygen supply, through the oxygen tube, out the oxygen delivery device, and to the patient or person.

Since oxygen tanks and oxygen concentrators are generally quite cumbersome, most patients use a long oxygen hose or a series of oxygen hoses coupled together so that they may move about the room or the house without bringing the oxygen supply with them. The patient merely moves throughout the room or house while wearing the oxygen mask or nasal cannula and the oxygen hose follows them.

Sometimes, however, an oxygen hose may get caught or pinched in something like a doorframe or a piece of furniture. This can lead to the oxygen hose becoming disconnected at the oxygen supply, at any point where two hoses are coupled together, or at the connection to the oxygen mask or nasal cannula. If the oxygen hose becomes disconnected, the oxygen flow to the oxygen delivery device will cease.

The oxygen flow is typically only supplemental and flows at a very low rate which can sometime be unnoticeable to the patient or person. At such a low flow rate, a patient or person is likely to not notice if the oxygen flow out of the oxygen delivery device ceases for any reason. Accordingly, a patient may go without oxygen for a period of time without being aware that the oxygen flow has stopped which may lead to significant adverse effects on their health.

Accordingly, it is desirable to have a device which may alert a patient or caretaker if the oxygen hose is disconnected at any point along its length.

Additionally, it may be desirable for such a device to have a slim profile matching that of the oxygen tube such that the device does not increase the likelihood of the oxygen hose being disconnected.

Lastly, it may be desirable for such a device to have a low cost for manufacture so that the device may be single use or frequently replaced.

### SUMMARY OF THE INVENTION

In general, in a first aspect, the invention relates to an oxygen hose disconnection alert device comprising a tubular body having a first end and a second end; a first oxygen line connector on the first end; a second oxygen line connector on the second end; at least one sensor on the first end that detects connection of the first oxygen line connector to an oxygen line; and at least one sensor on the second end that detects connection of the first oxygen line connector to an oxygen line, where the oxygen hose disconnection alert device is capable of attempting to alert a user if the oxygen hose disconnection alert device is disconnected from an oxygen line.

The first oxygen line connector and/or the second oxygen line connector may comprise a barbed oxygen hose fitting.

The oxygen hose disconnection alert device may further comprise a battery.

The oxygen hose disconnection alert may further comprise an alert device. The alert device may comprise a buzzer, an LED light, a Bluetooth chip, a Wi-Fi card, or any combination thereof.

The at least one sensor may comprise a plunger switch, a pressure switch, a light detector, or any combination thereof.

### BRIEF DESCRIPTION OF THE DRAWINGS

FIG. 1 is a side perspective view of one embodiment of the oxygen hose disconnection alert device;

FIG. 2 is a top perspective view of the embodiment of the oxygen hose disconnection alert device as shown in FIG. 1;

FIG. 3 is a cutaway view showing the general circuitry of the embodiment of the oxygen hose disconnection alert device as shown in FIG. 1;

FIG. 3a is a section of the embodiment of the oxygen hose disconnection alert device as shown in FIG. 3 showing another portion of the general circuitry inside the device;

FIG. 4 is a top perspective view of an alternate embodiment of the oxygen hose disconnection alert device with the covers removed;

FIG. 5 is a top view of the oxygen hose alert device as shown in FIG. 4;

FIG. 6 is an alternate angle top perspective view of the oxygen hose alert device as shown in FIG. 4;

FIG. 7 is a bottom perspective view of the oxygen hose alert device as shown in FIG. 4;

FIG. 8 is a side view of the oxygen hose alert device as shown in FIG. 4;

FIG. 9 is a front view of the oxygen hose alert device as shown in FIG. 4; and

FIG. 10 is an opposite side view of the oxygen hose alert device as shown in FIG. 4.

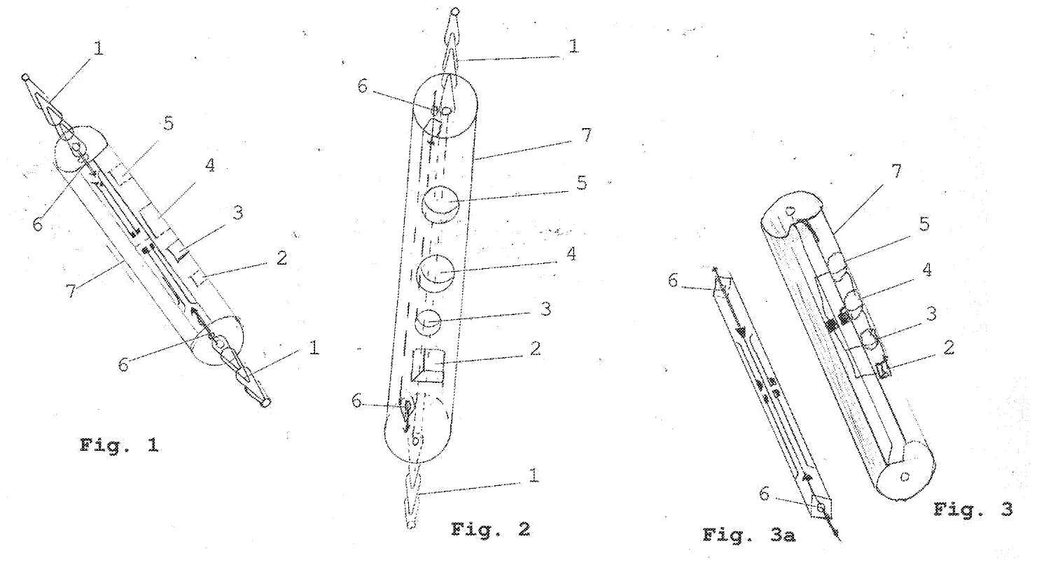

Other advantages and features will be apparent from the following description and from the claims.

### DETAILED DESCRIPTION OF THE INVENTION

The devices and methods discussed herein are merely illustrative of specific manners in which to make and use this invention and are not to be interpreted as limiting in scope.

While the devices and methods have been described with a certain degree of particularity, it is to be noted that many modifications may be made in the details of the construction and the arrangement of the devices and components without departing from the spirit and scope of this disclosure. It is understood that the devices and methods are not limited to the embodiments set forth herein for purposes of exemplification.

In general, in a first aspect, the invention relates to an oxygen hose disconnection alert device comprising a hollow cylindrical body 7 that may have barbed hose connection fittings 1 disposed on each end. Constructed this way the device's general structure is the same as that of an oxygen hose coupling device. It is preferable that the cylindrical body may have as close to the same radius as a standard oxygen hose.

The oxygen hose disconnection alert device may have a mechanism for detecting if an oxygen hose is connected to each of the barbed hose connection fittings 1. In one embodiment of the present invention, the mechanism is at least one plunger switch 6 located at each end of the cylindrical body 7. The plunger switch may be connected to a battery 4 and an alert device which may be embedded inside the cylindrical body 7. The battery may be permanent.

The alert device may be any device which can alert a patient or a caretaker that the oxygen hose has been disconnected. The embodiment shown in the figures includes a buzzer 5, an LED light 3, and a Bluetooth chip 2. The circuitry of the embodiment shown in the figures is designed such that when the plunger switches 6 are pressed (which would indicate that an oxygen hose is connected to either end) the battery 4 does not power the buzzer 5, LED light 3, or Bluetooth chip 2. If either plunger switch 6 is released or both plunger switches 6 are released, the circuit is closed. When the circuit closes, the buzzer 5 emits a sound, the LED light 3 illuminates, and the Bluetooth chip 2 emits a signal. The emitted signal may be received by a separate device such as a cell phone or computer which may then alert a patient or caretaker that the oxygen hose has been disconnected. Even further, the Bluetooth signal may be read by a mobile application on the patient's phone that, upon receipt of said signal, notifies the patient and anyone else (such as a caretaker) it is programmed to notify.

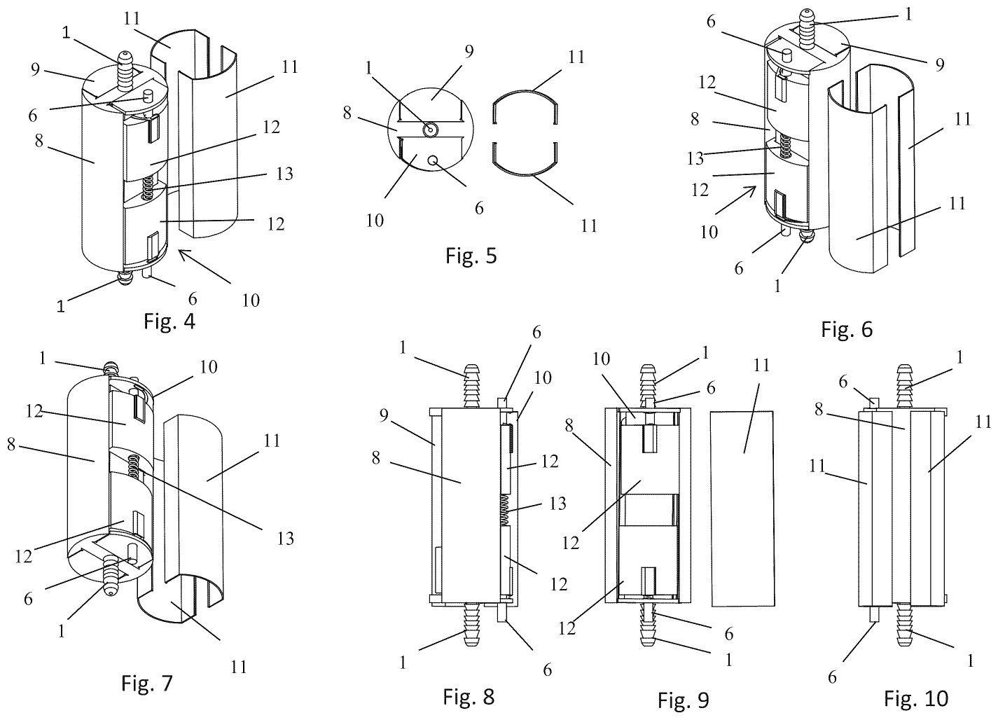

In an alternate embodiment, shown in FIGS. 4 through 10, the barbed hose connection fittings 1 disposed on each end of a main body 8. Each of the fittings 1 may swivel or be fixed, such that both may swivel, both may remain fixed, or one side may swivel while the other side may remain fixed. The main body 8 may have a nickel platted SS tube for airflow with FDA approved nipple slide connectors. The main body 8 may have opposing recesses capable of housing a communication component 9 on one side and a battery component 10 on the other side. The communication component 9 may be capable of Bluetooth communication and may additionally or alternately house the buzzer and/or LED light described above and/or any other desired communication device. The communication component 9 may be in communication with and powered by the battery component 10. Both the communication component 9 and the battery component 10 may snap, slide, and/or lock into place relative to the main body 8. Covers 11 may snap on or otherwise attach to either side of the main body 8 to cover the communication component 9 and the battery component 10 independently.

The battery component 10 may house a pair of sliding switches 12, which may be connected via a metal plate on top of the battery negative side. A spring 13 may tend to push the sliding switches 12 apart, one toward the upper end of the device and one toward the lower end of the device. Each of the sliding switches 12 may have a soft plunger 6, one projecting from the upper end of the device and one projecting from the lower end of the device. The plungers 6 may be located such that they are depressed when oxygen tubes correctly attached to the barbed hose connection fittings 1.

As with the first embodiment, the circuitry of the alternate embodiment may be designed such that when the plungers 6 are pressed (which would indicate that an oxygen hose is connected to either end) the battery component 10 does not power the communication component 9, but if either plunger 6 is released or both plungers 6 are released, the circuit is closed. When the circuit closes, the communication component 9 may be powered by the batter component 10. As a result, the buzzer may emit a sound, the LED light may illuminate, and/or the Bluetooth chip may emit a signal. The emitted signal may be received by a separate device such as a cell phone or computer which may then alert a patient or caretaker that the oxygen hose has been disconnected. Even further, the Bluetooth signal may be read by a mobile application on the patient's phone that, upon receipt of said signal, notifies the patient and anyone else (such as a caretaker) it is programmed to notify.

While the preferred embodiment comprises a buzzer, an LED light, and a Bluetooth chip, other alert devices such as Wi-Fi chips or any combination of the aforementioned may be used.

Further, while the preferred embodiment comprises a set of plunger switches, any other mechanism for detecting oxygen hose connection such as contact switches, pressure switches, light detectors, or any similar device may be used.

Lastly, the cylindrical body may only have a barbed hose connection fitting on one end such that one side connects to an oxygen hose while the other connects to an oxygen supply source which may require a different fitting. Further, the device may detect and alert whether the device is connected to an oxygen supply source similar to how it detects and alerts whether it is connected to an oxygen hose.

Whereas the devices and methods have been described in relation to the drawings and claims, it should be understood that other and further modifications, apart from those shown or suggested herein, may be made within the spirit and scope of this invention.

What is claimed is:

1. An oxygen hose disconnection alert device comprising:
a tubular body having a first end and a second end;
a first oxygen line connector on the first end;
a second oxygen line connector on the second end;
at least one sensor on the first end that detects connection of the first oxygen line connector to an oxygen line; and
at least one sensor on the second end that detects connection of the first oxygen line connector to an oxygen line,
where the oxygen hose disconnection alert device is capable of attempting to alert a user if the oxygen hose disconnection alert device is disconnected from an oxygen line.

2. The oxygen hose disconnection alert device of claim 1 where the first oxygen line connector and/or the second oxygen line connector comprises a barbed oxygen hose fitting.

3. The oxygen hose disconnection alert device of claim 1 further comprising a battery.

4. The oxygen hose disconnection alert device of claim 1 further comprising an alert device.

5. The oxygen hose disconnection alert device of claim 4 where the alert device comprises a buzzer, an LED light, a Bluetooth chip, a Wi-Fi card, or any combination thereof.

6. The oxygen hose disconnection alert device of claim 1 where the at least one sensor comprises a plunger switch, a pressure switch, a light detector, or any combination thereof.
